(19) United States
(12) Patent Application Publication
(10) Pub. No.: US 2017/0197088 A1
(43) Pub. Date: Jul. 13, 2017

(54) USE OF POLYMERIZING MATERIALS TO ACHIEVE EN BLOC RESECTION

(71) Applicant: Boston Scientific Scimed, Inc., Maple Grove, MN (US)

(72) Inventor: John A. Hingston, Framingham, MA (US)

(21) Appl. No.: 15/402,029

(22) Filed: Jan. 9, 2017

Related U.S. Application Data
(60) Provisional application No. 62/276,535, filed on Jan. 8, 2016.

(51) Int. Cl. A61N 5/06; A61B 1/31; A61K 41/00; A61B 18/24; A61M 5/32; A61M 25/00; A61B 1/018; A61B 1/015

(52) U.S. Cl. CPC A61N 5/062; A61B 1/018; A61B 1/31; A61B 1/015; A61B 18/24; A61M 5/3294; A61M 25/0084; A61K 41/0057; A61B 2017/0034

(57) ABSTRACT

This disclosure concerns systems and methods for tissue resection. Systems according to the various embodiments of the invention include cutting instruments, particularly laser cutting heads, and/or polymer materials that form polymer bodies when flowed into tissue.

### CROSS-REFERENCE TO RELATED APPLICATIONS

This application claims the benefit of priority to U.S. Provisional Application Ser. No. 62/276,535, filed Jan. 8, 2016, the disclosure of which is herein incorporated by reference in its entirety.

### FIELD OF THE INVENTION

This application relates to the field of medical devices and medical procedures.

More particularly, the application is related to devices and methods for tissue bulking and tissue resection.

### BACKGROUND

Colorectal cancer is among the most common cancers, and is responsible for approximately 60,000 fatalities each year in the United States alone. (Ahmedine Jamal, et al., Cancer Statistics 2002, CA: A Cancer Journal for Clinicians, Volume 52, pp 23-47 (2002)). Early detection and treatment of these cancers is critical for patient survival: five year survival rates are about 90% among those with localized tumors, but are less than 10% for patients with metastases. (Robert A. Smith et al., Colorectal Cancer (in Holland-Frei Cancer Medicine, 6th Ed., Kufe et al., eds. (2003))). The American Cancer Society recommends periodic screening for colon cancer among older adults, but fewer than 30% of older adults have ever been screened. (Id.)

Colonoscopy is a particularly useful colon cancer screening method, which involves the insertion of a specialized endoscope (a “colonoscope”) into the large bowel to permit direct visual inspection of the lower digestive tract and, if necessary, resection of small tumors (also referred to as “polyps”). In addition, colonoscopy is frequently employed if another screening method (such as detection of occult blood in the stool or sigmoidoscopy) suggests that cancer may be present. However, polyp resection (“polypectomy”) using colonoscopy requires a high degree of skill and training, and may accordingly be costly and time consuming.

In a typical colonoscopic polyp resection (“polypectomy”) procedure, the endoscopist must dissect the polyp (which may be as small as several millimeters in diameter) away from the healthy intestinal mucosa surrounding it, while minimizing penetration of the underlying submucosal tissue, which may result in damage or perforation of the intestine. To facilitate the procedure, the endoscopist may inject saline beneath the polyp to help separate it from the submucosal tissue, however the saline will dissipate rapidly once cutting begins. Thus, current methods may facilitate the separation of the polyp from the submucosa at the beginning of the resection process, but not at the end. A more durable separation between polyp and submucosa, if achieved, could further simplify polyp resection significantly while reducing the risk of bowl perforation or submucosal damage.

### SUMMARY OF THE INVENTION

The present invention, in its various aspects, provides improved systems and methods for tissue bulking and resection of tissue layers, or growths confined to a tissue layer, while sparing underlying tissue layers. For instance, the present invention provides systems and method for polypectomy in which polyps are more effectively separated from submucosal tissue layers.

In one aspect, the present invention relates to a system for resecting a patient's tissue, which includes (a) a flowable methacrylate polymer fluid that is configured to (i.e. that reacts to) form a solid body when disposed between first and second layers of the tissue, (b) a needle for disposing the flowable methacrylate polymer between the first and second tissue layers, and (c) a cutting device insertable into the body of the patient. At least one of the needle and the cutting device is insertable through a working channel of an endoscope. In various embodiments, the methacrylate polymer includes one or more of a pore, a bubble, a pigment, and a particle comprising a metal; if the cutting device includes a laser, then such pore, bubble, pigment or particle may have a high reflectance or absorbance of a wavelength emitted by the laser; such features can also, optionally, improve the radiopacity of the resulting polymer bodies to facilitate imaging. In some cases, the flowable methacrylate polymer is formed by mixing the first and second components, for example a first component that is a fluid comprising a methyl methacrylate monomer and/or a second component including polymethylmethacrylate prepolymer in solid form; this mixing happens, variously, within the body and/or within a mixing element such as a static mixer to which the needle is fluidly connected. One or both of these components can include a particle comprising a metal, a pigment, an entrained gas, a porogen and/or a material that liberates gas when the first and second components are mixed or disposed between the tissue layers. In some cases, the cutting instrument includes a housing comprising a plurality of waveguides connectable to a laser source, the waveguides arranged in a line. The cutting instrument also optionally includes one or more of at least one skid disposed along an exterior surface of the housing so as to separate the housing from a surface of the tissue and to orient the plurality of waveguides in parallel to the surface of the tissue and/or a blade formed integrally with the housing, which blade is parallel to the plurality of waveguides and, wherein such skid or blade optionally or additionally, defines at least one flat surface parallel to a flat exterior surface of the housing, is reversibly attachable to the housing, and/or includes a material that transmits light. The systems according to this aspect of this invention are useful in medicine, particularly in procedures for resection of colon polyps.

In another aspect, the present invention relates to a method of resecting a polyp in the colon of a patient that includes inserting a colonoscope into the patient's colon, flowing, through an instrument extended through a working channel of the colonoscope, a polymer formulation configured to cross-link or polymerize into a tissue proximate the polyp, thereby forming a polymer body between the polyp and a submucosal tissue, and resecting the polyp. In various cases, the polymer formulation includes polymethylmethacrylate and/or a material that refracts or absorbs light such as a metal flake or particle, a quantum dot or a pigment. The method can include applying laser energy to the tissue near the polyp and overlying the polymer body, for example by means of a steerable laser device inserted through a working channel of the colonoscope. In some cases, the laser is applied using a device comprising a plurality of linearly-arranged waveguides (and, optionally, a mechanical cutting apparatus such as a blade disposed near the tissue-contacting surface of the device) and the method includes delivering laser energy through at least one of the plurality of linearly-arranged waveguides, and advancing the laser device toward the polyp. In some cases, the polymer body is a foam. Alternatively, non-laser cutting instruments such as cryoablation, electrocautery, and mechanical cutting devices can be used to resect the tissue in an analogous manner.

In yet another aspect, the present invention relates to a method of resecting a tissue layer that includes forming a polymethylmethacrylate (PMMA) body between first and second tissue layers and applying an effective amount of laser energy to the first tissue layer to separate a portion of the first tissue layer, wherein the PMMA body absorbs or reflects a portion of the laser energy, thereby sparing the second tissue layer underlying the PMMA body. As described above, the PMMA body can include one or more of a pigment, metal particle, bubble or pore. The method can be applied to remove or resect tissue layers throughout the body, including mucosal tissue or mucosal structures such as polyps in the digestive tract (e.g. the stomach, small intestine, large intestine), fibroids in the reproductive tract (including the uterus and connected anatomy), and strictures throughout the body, including the urethra, ureter, esophagus, etc.

In yet another aspect, the present invention relates to systems and methods for tissue bulking by forming polymer bodies within tissue walls and/or between tissue layers as described above.

### DRAWINGS

Aspects of the invention are described below with reference to the following drawings in which like numerals reference like elements, and wherein:

FIGS. 1A through 1D show schematic views of a polyp resection apparatus according to certain embodiments of the present invention.

FIG. 2A through 2E shows a schematic view of a polyp resection method according to certain embodiments of the present invention.

Unless otherwise provided in the following specification, the drawings are not necessarily to scale, with emphasis being placed on illustration of the principles of the invention.

### DETAILED DESCRIPTION OF THE PREFERRED EMBODIMENTS

In general, the various systems and methods described herein utilize materials that polymerize in situ to create a mechanical and/or optical barrier between a polyp or other mucosal growth and the underlying submucosa, reducing the risk of perforation during resection. Also described are novel resection tools that can be used in conjunction with polymerizing materials to simplify polypectomy procedures.

### Laser Incision Devices for En Bloc Resection

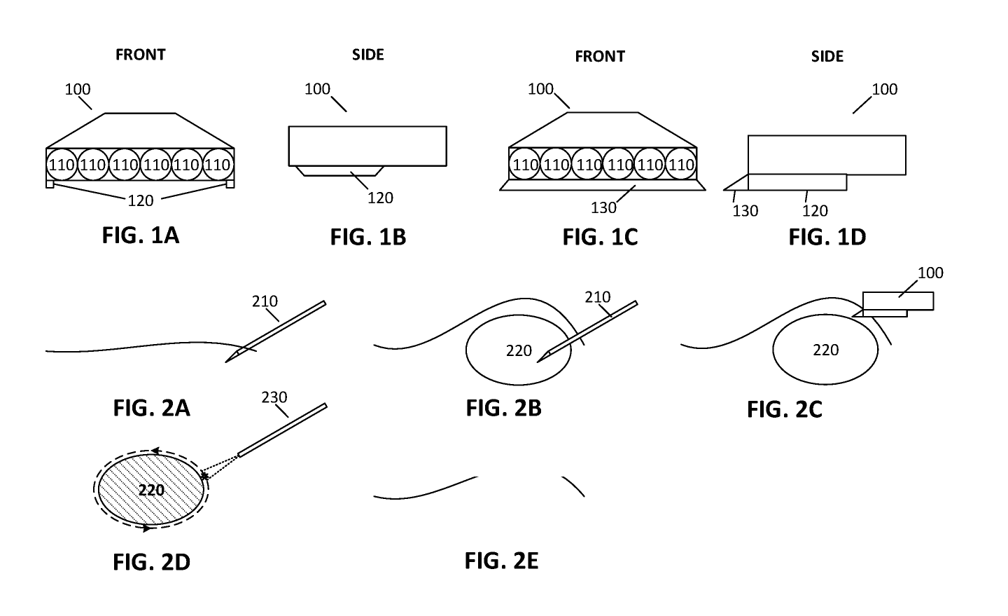

Turning to FIGS. 1A through 1D, in one set of embodiments, the present disclosure relates to linear laser incision devices that are configured to move and, optionally, mechanically incise, along a mucosal surface, simplifying resection of polyps. FIGS. 1A and 1B illustrate one embodiment of a laser cutting head 100 which includes a plurality of waveguides 110 arranged linearly therewithin and one or more skids 120 that minimize friction between the underside of the cutting head 100 and the mucosal surface and allow it to be advanced smoothly and uniformly. FIGS. 1C and 1D illustrate a similar arrangement, in which the underside of the cutting head 100 includes a mechanical cutting implement 130, such as a linear blade. In these embodiments, skids 120 are also optionally used as shown in FIG. 1D, but friction may also be reduced by the application of a lubricious coating to the underside of the cutting head 100. The waveguides 110 may be narrow gauge (e.g. 0.3 mm) optical fibers that transmit laser wavelengths useful for resection. For instance, the fibers may transmit mid infrared wavelengths, enabling the use of mid-infrared laser excision, which advantageously results in cauterization of incisions as they are made, reducing the extent of bleeding. Alternatively, the fibers may transmit near infrared, visible and/or ultraviolet wavelengths, enabling plasma excision, which can be highly precise. Adding to the precision of the excision, the cutting head preferably delivers a narrow beam, for instance of less than 1 mm (e.g. 0.3 mm, 0.4 mm, 0.5 mm, etc.). The cutting head also preferably has a relatively short focal distance (e.g. 0.5 mm), so that the laser energy delivered to surrounding tissue (and, consequently, the potential for damage to surrounding tissue) is minimized.

In use, an operator will identify a polyp or other structure to be resected along the wall of the colon, and will preferably (though not necessarily) inject saline or a polymerizing composition as described below into the polyp to separate it from the underlying submucosa and facilitate resection. Thereafter, the user may position the cutting head 100 next to the polyp and activate a laser source to deliver laser energy to the tissue and separate the polyp from the surrounding mucosa. The user will also generally advance the cutting head forward to continue cutting the polyp. When a cutting head 100 with a cutting blade 130 is utilized, the user may advance the cutting head 100 to excise the polyp without activating the laser source if that is desired. The cutting blade 130 is optionally electroactive or “hot,” so as to concomitantly sever and cauterize the tissue.

The cutting head according to the embodiments presented above has several advantages over currently-used laser resection tools: by allowing a user to slide (and brace) the cutting head 100 against the tissue surface, more precise cuts are possible than those made when the cutting head is unbraced and freely movable in space. Cleaner, more precise cuts will, in turn, reduce blood contamination of pathology samples. In addition, cutting heads according to the designs presented above may require less skill to use than current laser resection tools.

### Polymethylmethacrylate (PMMA) Compositions for En Bloc Resection

Turning next to FIG. 2A-2E, in certain embodiments of the present invention, a polymerizing or cross-linking composition is injected beneath a polyp or other structure to be resected to form a stable, solid or semi-solid body separating the polyp or structure from the underlying submucosa and, optionally, providing a physical and/or optical barrier between the polyp and underlying tissue. In cases where the polymerization or cross-linking reaction requires water, or proceeds normally in aqueous solution, the polymer is injected into a fluid-filled space created by the injection of saline below the polyp. In preferred embodiments, a two-part PMMA formulation is injected into the tissue using a needle 210, then undergoes a self-polymerization reaction in situ to form a polymer body 220 beneath the polyp. PMMA ‘cures’ by free radial polymerization. The composition consists of two parts: Part A is a powder comprising pre-polymerized PMMA particles and, optionally, one or more of an antibiotic (such as gentamycin, if infection is a concern) and a particulate radiopaque material to facilitate visualization of the resulting polymer body 220. Part B is a fluid phase comprising methyl methacrylate (MMA) monomer, a stabilizer and an inhibitor. These components are optionally pre-mixed immediately prior to deployment beneath a polyp (e.g. by means of a mixing nozzle, or by mixing the two components and feeding them into a delivery device), though in some instances the two components are injected directly into the tissue wall, where they mix and form a solid PMMA body 220 which elevates the polyp or other structure into the lumen of the colon and away from the underlying submucosa. In addition to remaining in place during the cutting process (in contrast to currently used saline or other fluid bulking materials which dissipate once cutting begins) the polymer body 220 can also advantageously form a stable surface for a cutting instrument such as the cutting head 100 described above or, as shown in FIG. 2D, a steerable laser device 230, such as a steerable near infrared laser device.

The polymer body 220 will act as a physical barrier during an excision procedure and may also act as an optical barrier to prevent the delivery of laser energy to tissue underlying the polymer body. For example, a PMMA polymer body may absorb ultraviolet wavelengths, while metallic solids (such as flakes, particles, etc. added to the polymer, can improve the absorption or reflection of laser energy by the polymer body 220 to prevent damage to surrounding tissue. The skilled artisan will appreciate that a variety of materials, including quantum dots and pigments (which are absorptive) and pores or microbubbles, etc. (which are diffractive), can be utilized to improve the optical shielding characteristics of the polymer body 220.

The polymer formulation can also include a gas source such as entrained gas or a reactant that liberates gas, in which case the resulting polymer body 220 will be a polymer foam. In some embodiments, the gas is CO₂ gas, which may be liberated from a chemical reaction that occurs before or during formation of the polymer body 220. One exemplary method for forming a polymer foam is described by Forest et al., CO₂ nano-foaming of nanostructured PMMA, Polymer vol. 58, issue 10, pp. 76-87 (Feb. 10, 2015).

In use, the polymer body 220 remains intact and in place once delivered, and functions to separate a polyp or other superficial tissue layer from underlying tissue layers. In contrast to bulking using saline, as is currently done, the polymer body 220 will not dissipate during the cutting process, but will remain intact; once resection is complete, the polymer body 220 will be unconstrained and able to pass through the colon and, eventually, be expelled by the body.

### CONCLUSION

It should be appreciated that, although the foregoing examples have focused on resection of colonic polyps, the systems and methods of the present invention can be adapted for endoscopic resection of other tumors, such as uterine fibroids.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “consists essentially of” means excluding other materials that contribute to function, unless otherwise defined herein. Nonetheless, such other materials may be present, collectively or individually, in trace amounts.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present invention have described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

1. A method for resecting a polyp in a colon of a patient, comprising the steps of:
inserting, into the colon of the patient, a colonoscope;
flowing, through an instrument extended through a working channel of the colonoscope, a polymer formulation configured to cross-link or polymerize into a tissue proximate the polyp, thereby forming a polymer body between the polyp and a submucosal tissue; and
resecting the polyp.

2. The method of claim 1, wherein the polymer formulation includes polymethylmethacrylate.

3. The method of claim 1, wherein the polymer formulation includes a material that refracts or absorbs light.

4. The method of claim 3, wherein the material is a metal flake or particle.

5. The method of claim 3, wherein the material is a quantum dot or a pigment.

6. The method of claim 3, wherein the step of resecting the polyp includes applying laser energy to the tissue near the polyp and overlying the polymer body.

7. The method of claim 6, wherein the step of applying laser energy to the tissue includes delivering the laser energy using a steerable laser device inserted through a working channel of the colonoscope.

8. The method of claim 6, wherein the step of applying laser energy includes contacting a portion of the colon near the polyp with a surface of a laser device comprising a plurality of linearly-arranged waveguides, delivering laser energy through at least one of the plurality of linearly-arranged waveguides, and advancing the laser device toward the polyp.

9. The method of claim 8, wherein the laser device includes a mechanical cutting apparatus.

10. The method of claim 9, wherein the mechanical cutting apparatus is a blade disposed near the surface of the laser device.

11. The method of claim 1, wherein the polymer body is a polymer foam.

12. A system for resecting a tissue of a patient, comprising:
a methacrylate polymer fluid configured to form a polymer body when disposed between first and second layers of the tissue;
a needle for disposing the flowable methacrylate polymer between the first and second layers of the tissue; and
a cutting device insertable into the body of the patient,
wherein at least one of the needle and cutting device is insertable through a working channel of an endoscope.

13. The system according to claim 12, wherein the methacrylate polymer fluid includes one or more of a pore, a bubble, a pigment, and a particle comprising a metal.

14. The system according to claim 13, wherein the cutting device includes a laser and the pore, bubble, pigment or particle has a high reflectance or absorbance of a wavelength emitted by the laser.

15. The system according to claim 12, wherein the flowable methacrylate polymer fluid is formed by mixing first and second components.

16. The system according to claim 15, wherein the first component is a fluid comprising methyl methacrylate monomer.

17. The system according to claim 15, wherein the second component includes a polymethylmethacrylate pre-polymer in solid form.

18. The system according to claim 15, wherein at least one of the first and second components includes a particle comprising a metal, a pigment, an entrained gas, and a material that liberates gas when the first and second components are mixed or disposed between the tissue layers.

19. A method of resecting a tissue layer, comprising the steps of:
forming a polymethylmethacrylate (PMMA) body between first and second tissue layers; and
applying an effective amount of laser energy to the first tissue layer to separate a portion of the first tissue layer, wherein the PMMA body absorbs or reflects a portion of the laser energy, thereby sparing the second tissue layer underlying the PMMA body.

20. The method according to claim 19, wherein the PMMA body includes one or more of a pigment, metal particle, bubble or pore.
